(19) United States
(12) Patent Application Publication
(10) Pub. No.: US 2007/0144968 A1
(43) Pub. Date: Jun. 28, 2007

(54) Separation of fulvestrant isomers

(76) Inventors: Cristian Fazioni, Lentate sul Seveso (MI) (IT); Andrea Giolito, Milan (IT)

Correspondence Address: KENYON & KENYON LLP, ONE BROADWAY, NEW YORK, NY, 10004, US

(21) Appl. No.: 11/544,522

(22) Filed: Oct. 5, 2006

Related U.S. Application Data
(60) Provisional application No. 60/724,059, filed on Oct. 5, 2005.

(51) Int. Cl. B01D 15/08

(52) U.S. Cl. 210/635; 210/656; 436/161

(57) ABSTRACT

The invention encompasses methods of separating the isomers of fulvestrant comprising placing a fulvestrant sample on a HPLC using a reverse phase column or chiral column; eluting the sample with an eluant having a first mobile phase and a second mobile phase; and collecting purified fractions of fulvestrant sulfoxide A or fulvestrant sulfoxide B from the column. The method provides fulvestrant sulfoxide A or fulvestrant sulfoxide B in 99.5% purity as determined by HPLC.

### RELATED APPLICATIONS

This application claims the benefit of U.S. provisional application Ser. No. 60/724,059, filed on Oct. 5, 2005.

### FIELD OF THE INVENTION

The invention encompasses methods of separating diastereomers of fulvestrant using reverse phase and chiral HPLC systems and the diastereomerically pure fulvestrant sulfoxide A and fulvestrant sulfoxide B produced by the methods.

### BACKGROUND OF THE INVENTION

Many breast cancers have estrogen receptors (ER) and the growth of these tumors can be stimulated by estrogen. Fulvestrant is an estrogen receptor antagonist that binds to the estrogen receptor in a competitive manner with affinity comparable to that of estradiol. Fulvestrant down regulates the EP protein in human breast cancer cells. The chemical name of fulvestrant is 7-α-[9-(4,4,5,5,5,-pentafluoropentylsulphinyl)nonyl]estra-1,3,5-(10)-triene-3,17-β-diol and it has the following chemical structure:

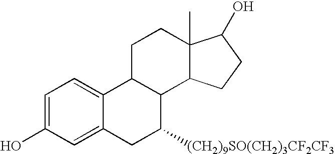

Fulvestrant is commercially available under the name FASLODEX®. In a clinical study in postmenopausal women with primary breast cancer treated with single doses of FASLODEX® 15-22 days prior to surgery, there was evidence of increasing down regulation of ER with increasing dose. This was associated with a dose-related decrease in the expression of the progesterone receptor, an estrogen-regulated protein. These effects on the ER pathway were also associated with a decrease in Ki67 labeling index, a marker of cell proliferation.

Fulvestrant exists as a mixture of two diastereomers which are epimeric at the sulphur atom of the side chain. These two diastereomers are known as Fulvestrant Sulfoxide A and Fulvestrant Sulfoxide B.

No synthetic route for the synthesis of one pure diastereomer is described in the literature or in the proposed process. The present invention proposes to solve this need by providing a method for efficiently separating the diastereomers of fulvestrant.

### SUMMARY OF THE INVENTION

One embodiment of the invention encompasses a method of detecting fulvestrant diastereomers comprising placing a fulvestrant sample on a HPLC using a reverse phase system; eluting the sample with two mobile phases using a non-linear gradient having a first mobile phase and a second mobile phase; and detecting the separate isomers by HPLC, wherein the first mobile phase is water or an aqueous buffer and the second mobile phase is acetonitrile, tetrahydrofuran, or methanol. The fulvestrant sample may be a mixture of fulvestrant sulfoxide A and fulvestrant sulfoxide B, such as a racemic mixture or a mixture enhanced in either fulvestrant sulfoxide A and fulvestrant sulfoxide B. The packing material of the reverse phase column may be C8 (octyl), C18 (octadecyl), phenyl, pentafluorophenyl, or phenylhexyl and preferably, C8 (octyl) or C18 (octadecyl). In the method, the first mobile phase has an initial amount of about 40% to about 70% by volume, and the second mobile phase has an initial amount of about 30% to about 60% by volume. Preferably, the first mobile phase has a final amount of about 40% to about 0% by volume, and the second mobile phase has a final amount of about 100% to about 50% by volume.

Another embodiment of the invention encompasses a method of separating fulvestrant diastereomers comprising placing a fulvestrant sample on a HPLC having a chiral column system; eluting the sample with two mobile phases using an isocratic solvent system having a first mobile phase and a second mobile phase; and collecting purified fractions of fulvestrant sulfoxide A or fulvestrant sulfoxide B from the column, wherein the first mobile phase is at least one C₅-C₁₀ alkane and the second mobile phase is a C₃ alcohol.

The packing material of the chiral column may be amylose tris(3,5-dimethylphenylcarbamate), β-cyclodextrin, cellobiohydrolase, selector R-(−)—N-(3,5-dinitrobenzoyl)-phenylglycine, or cellulose tris(3,5-dimethylphenylcarbamate) and preferably, the packing material of the chiral column is amylose tris(3,5-dimethylphenylcarbamate). The column may have a packing particle of a size of about 3 μm to about 10 μm and preferably, the column has a packing particle a size of about 5 μm. Preferably, when using a chiral column system, the first mobile phase is n-hexane, and the second mobile phase is isopropanol. The first mobile phase may be present in an amount of about 75% to about 95% by volume and the second mobile phase is present in an amount of about 5% to about 25% by volume. Preferably, the first mobile phase is present in an amount of about 85% by volume and the second mobile phase is present in an amount of about 15% by volume.

The method of separating fulvestrant diastereomers using the chiral column may further comprise crystallizing fulvestrant sulfoxide A or fulvestrant sulfoxide B from the purified fractions by dissolving fulvestrant sulfoxide A or fulvestrant sulfoxide B in organic solvent to form a mixture and precipitating from the mixture fulvestrant sulfoxide A or fulvestrant sulfoxide B. Typically, the organic solvent is ethyl acetate or toluene. The mixture may be heated to reflux followed by cooling to a temperature of about 0° C. to about 25° C., preferably the mixture is cooled to a temperature of about 4° C.

Yet another embodiment of the invention encompasses fulvestrant sulfoxide A or fulvestrant sulfoxide B that is 99.5% isomerically pure as determined by HPLC.

### BRIEF DESCRIPTION OF THE FIGURES

FIG. 1 illustrates the HPLC chromatogram of fulvestrant as obtained in Example 1.

FIG. 2 illustrates the HPLC chromatogram of fulvestrant as obtained in Example 2.

FIG. 3 illustrates an HPLC chromatogram for Sulfoxide A as obtained in Example 3.

FIG. 4 illustrates an HPLC chromatogram for Sulfoxide B as obtained in Example 3.

FIG. 5 illustrates the HPLC chromatogram of Sulfoxide A separated by the methodology of Example 3 and obtained using the HPLC methodology of Example 1.

FIG. 6 illustrates the HPLC chromatogram of Sulfoxide B separated by the methodology of Example 3 and obtained using the HPLC methodology of Example 1.

### DETAILED DESCRIPTION OF THE INVENTION

The invention encompasses methods of detecting and/or separating the isomers of fulvestrant. The method can be used to enrich or completely isolate one fulvestrant isomer. The methods may be used on a small or large scale, including preparation scale or industrial scale separation of the isomers. The method of separating fulvestrant sulfoxide isomers can be used in the preparation of fulvestrant sulfoxide standards, wherein the sulfoxide standard has one fulvestrant sulfoxide isomer. The standard can then be used to qualitatively or quantitatively determine the presence of fulvestrant sulfoxide A and/or fulvestrant sulfoxide B. Moreover, the method of the invention can be used to make pharmaceutical compositions of substantially isomerically pure fulvestrant.

The invention comprises methods of separating fulvestrant diastereomers by placing a fulvestrant sample on an HPLC system using either a reverse phase system or a chiral system with a column and two mobile phases. The selection of mobile phases is determined by the column system used, as described in greater detail below. One embodiment of the invention encompasses methods of detecting diastereomers of fulvestrant comprising placing a fulvestrant sample on a HPLC using a reverse phase system, eluting the sample with two mobile phases using a non-linear gradient having a first mobile phase and a second mobile phase, and detecting the separate isomers by HPLC, wherein the first mobile phase is water or an aqueous buffer and the second mobile phase is acetonitrile, tetrahydrofuran, or methanol. Another embodiment of the invention encompasses methods of separating diastereomers of fulvestrant comprising placing a fulvestrant sample on a HPLC having a chiral column system, eluting the sample with two mobile phases using an isocratic solvent system having a first mobile phase and a second mobile phase, and, collecting the separate isomeric fractions from the column, wherein the first mobile phase is at least one C₅-C₁₀ alkane and the second mobile phase is a C₃ alcohol.

Typically, the fulvestrant sample used as starting material in the method is a mixture of fulvestrant sulfoxide A and fulvestrant sulfoxide B. The mixture may be a racemic mixture or a mixture enhanced in one the two isomers, such as a 45:55 mixture of isomers. Thus, the fulvestrant sample may be crude fulvestrant such that the crude fulvestrant is purified and the isomers are separated. Alternatively, the fulvestrant sample may be purified fulvestrant, e.g., obtained after crystallization, such that the isomers are separated by using the above-described method. The fulvestrant used as the starting material in the separation can be made using methods disclosed in the art, such as U.S. Pat. No. 4,659,516, hereby incorporated by reference.

The column in the HPLC will determine the mobile systems used during the separation. In one embodiment, the invention comprises detecting fulvestrant diastereomers using a reverse phase column having solid support particles. Typically, the solid support particle is a silica derivative. Suitable silica derivatives include, but are not limited to, C8 (octyl), C18 (octadecyl), phenyl, pentafluorophenyl, or phenylhexyl. Preferably, the silica derivative is C8 (octyl) or C18 (octadecyl), such as the commercially available Alltima C18 by Alltech.

Alternatively, the column may be a chiral column. Typical chiral columns include, but are not limited to, amylose tris(3,5-dimethylphenylcarbamate), β-cyclodextrin, cellobiohydrolase, selector R-(−)—N-(3,5-dinitrobenzoyl)-phenylglycine, or cellulose tris(3,5-dimethylphenylcarbamate). Preferably, the chiral column is amylose tris(3,5-dimethylphenylcarbamate). Commercially available chiral columns include, but are not limited to, ChiraDex (Merck KGaA, Germany), Chiracell® OD (Daicel Chemical Industries, Ltd., Japan), Chiral-CBH (ChromTech, Ltd., UK), Bakerbond® DNBPG (covalent) (J.T. Baker, USA), and Chiralpak® AD-H (Daicel Chemical Industries, Ltd., Japan). The chiral column has a stationary packing material having the formula:

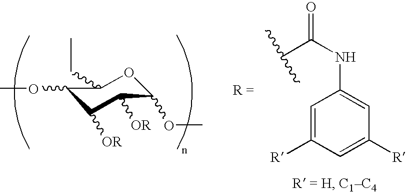

wherein “n” indicates a polymer. The length of the polymer may vary as included in the sample commercially available chiral columns described above.

The column packing particle typically has a size of about 3 μm to about 10 μm. Preferably, the column packing particle has a size of about 5 μm. The column length is typically about 100 mm to about 250 mm and a diameter of about 4.0 mm to about 20 mm.

The conditions for diastereomeric separation will depend upon whether the method uses a reverse phase column or a chiral column. Accordingly, each will be discussed separately below.

When using a reverse phase column, the eluant system is a non-linear gradient. In other words, the amount of each of the two mobile phases varies over time. Typically, the mobile phase is a two phase system comprising a first mobile phase and a second mobile phase. Typically, the first mobile phase is water or a buffered aqueous solution. Preferably, the first mobile phase is water. Buffered aqueous solutions suitable for the system include, but are not limited to, H₃PO₄ (Sol. 85%) 0.1% in water; trifluoroacetic acid 0.1% or 0.01% in water; formic acid 0.1% in water; phosphate buffer pH 3.2 (e.g. 7.2 g NaH₂PO₄ in 1800 mL of water, add 200 mL of a solution containing 2.5 g/mL of H₃PO₄ in water and if necessary, adjust the pH value and filter through a 0.2 μm membrane); or ion pair buffer (e.g. 2.9 g of sodium lauryl sulfate and 2.3 g of H₃PO₄ (Sol. 85%) in 1000 mL of water).

Typically, the second mobile phase is acetonitrile, tetrahydrofuran, or methanol. Preferably, the second mobile phase is acetonitrile. The first mobile phase can vary from an initial amount of about 40% to about 70% by volume, and preferably from an initial amount of about 50% to 60%. The first mobile phase can vary to a final amount of about 40% to about 0% by volume, and preferably, to a final amount of 30% by volume. The second mobile phase can vary from an initial amount of about 30% to about 60% by volume, and preferably, to an initial amount of about 40% to about 50% by volume. The second mobile phase can vary to a final amount of about 100% to about 50% by volume, and preferably, to a final amount of about 100% to about 70% by volume of the solvent mixture. More preferably, initially the eluant is 50% by volume of the first mobile phase and 50% of the second mobile phase, which is eluted for 60 minutes. Thereafter, the eluant is linearly changed to a mixture of 30% by volume of the first mobile phase and 70% of the second mobile phase for the next 40 minutes.

Typically, the reverse phase column temperature is about 10° C. to about 40° C., and preferably from about 15° C. to about 20° C. Typically, the flow rate is about 0.5 to about 1.5 ml/min, and preferably, about 0.5 ml/min to about 1.0 ml/min.

When using a chiral column, the eluant system is an isocratic system. In other words, the mobile phase comprises at least two solvents of fixed amounts that do not vary over time. The combination of solvents may be present as a mixture of solvents or as two mobile phases, a first mobile phase and a second mobile phase, that are combined at a fixed ratio. When the solvent system is a combination of mobile phases, then the first mobile phase is a C₅-C₁₀ alkane, and the second mobile phase is a C₃ alcohol, such as 1-propanol or 2-propanol. Preferably, the first mobile phase is n-hexane and/or heptane, and the second mobile phase is isopropanol. In the case wherein the solvent system is a combination of two mobile phases, then the phases two are combined in an amount of about 75% to about 95% of the first mobile phase and about 5% to about 25% of the second mobile phase by volume. Preferably, when the combined solvent system is about 85% of the first mobile phase and about 15% of the second mobile phase by volume. The typical amount of time for elution is about 45 minutes.

Typically, the chiral column temperature is from about 10° C. to about 40° C., and preferably the column temperature is about 30° C. to about 35° C. Typically, the flow rate is about 0.2 ml/min to about 5 ml/min. Preferably, the flow rate is about 0.6 to about 1.3 ml/min, and more preferably about 0.75 ml/min to about 0.9 ml/min.

The detector for the system can be any UV system that is commercially available. Typically, the detector is set to 220 nm and/or 240 nm.

The invention also encompasses crystallizing each of the fulvestrant diastereomers. Once each diastereomer is separated in the racemic mixture, and an oily residue is obtained after evaporation of the eluant phase, each diastereomer can be precipitated or crystallized from an organic solvent. Suitable organic solvents include, but are not limited to, ethyl acetate or toluene. Typically, the solvent is added to the residue and heated to reflux followed by cooling. Preferably, the heated solvent is cooled to about 0° C. to about 25° C., and more preferably, the heated solvent is cooled to about 4° C. The crystalline diastereomer may be collected by means commonly known to the skilled artisan, such as filtration. Thus, the process yields chromatographically pure solid fulvestrant sulfoxide A or fulvestrant sulfoxide B.

The processes described above can yield at least one of the diastereomers with an HPLC purity of greater or equal to about 99.5%.

Thus, another embodiment of the invention encompasses substantially isomerically pure fulvestrant Sulfoxide A or substantially isomerically pure fulvestrant Sulfoxide B. As used herein, unless otherwise defined, “substantially isomerically pure” means fulvestrant having more than 70% of one sulfoxide isomer as determine by HPLC area. Preferably, “substantially isomerically pure” means fulvestrant having more than 80% of one isomer as determine by HPLC area; more preferably, more than 90%; and even more preferably more than 95%. Most preferably, the term “substantially isomerically pure” means fulvestrant having more than 99% of one isomer as determine by HPLC area.

Another embodiment of the invention encompasses making internal or external standards of fulvestrant sulfoxide A or fulvestrant sulfoxide B using isomerically pure fulvestrant Sulfoxide A or substantially isomerically pure fulvestrant Sulfoxide B.

Furthermore, the process described above may be applied at an industrial scale using a Simulated Moving Bed system. This is suitable equipment for isocratic preparative purification. For example, it may be applied to pure fulvestrant having a mixture of sulfoxide A and sulfoxide B using a chiral system.

The invention also encompasses pharmaceutical compositions comprising substantially isomerically pure fulvestrant sulfoxide A or fulvestrant sulfoxide B, and a pharmaceutically acceptable excipient.

Having described the invention with reference to certain preferred embodiments, other embodiments will become apparent to one skilled in the art from consideration of the specification. The invention is further defined by reference to the following examples describing in detail the process of the invention. It will be apparent to those skilled in the art that many modifications, both to materials and methods, may be practiced without departing from the scope of the invention.

### EXAMPLES

### Example 1

### Gradient Reverse Phase HPLC Method

The separation was performed on an Agilent Technologies Mod. 1100 liquid chromatograph, equipped with a chiral column of C18 (250 mm×4.6 mm) having a 5 μm particle size (Alltima C18, Alltech). Two mobile phases were used in the HPLC unit. The first mobile phase was water and the second mobile phase was acetonitrile. The flow rate of eluant was set to 0.5 ml/minute, and the column temperature was set to 15° C. The test samples contained 1.0 mg/ml of fulvestrant in a solution of acetonitrile/methanol in a ratio of 50:50 by volume. The injection volume was 2 μl.

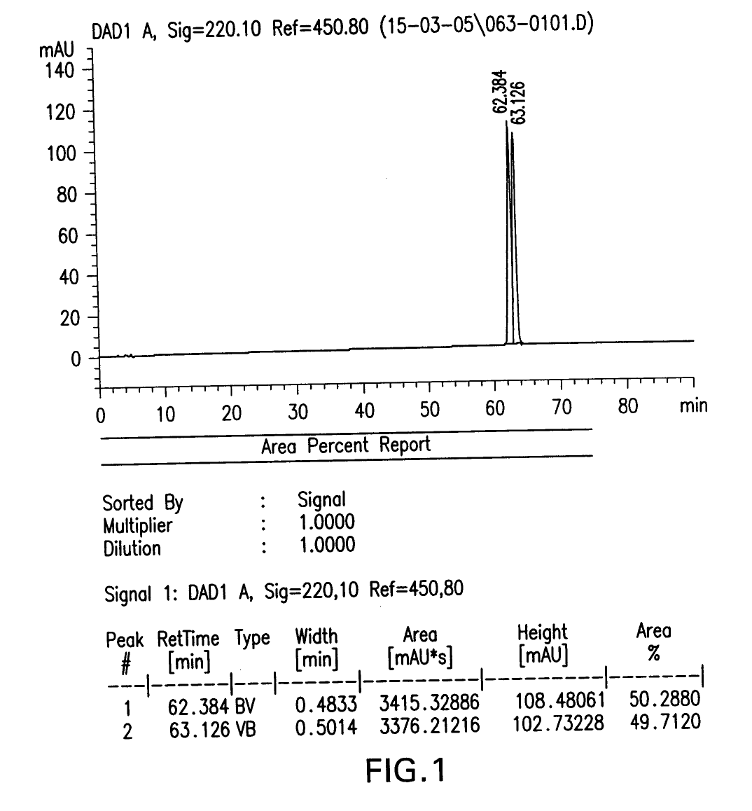

Initially, 50% of the first mobile phase and 50% of the second mobile phase were pumped through the system for 60 minutes (i.e., from time 0 to time 60 minutes). Thereafter, at after the 60 minutes to time 100 minutes, the composition of the eluant was changed in a linear fashion from 50% of the first mobile phase and 50% of the second mobile phase to 30% of the first mobile phase and 70% of the second mobile phase. The HPLC was equipped with a DAD detector at λ=220 nm with a bw=10 nm; and a reference signal =450 nm, bw=80 nm. The retention time of fulvestrant sulfoxide A was 62.4 min and the retention time of fulvestrant sulfoxide B was 63.1 min. FIG. 1 illustrates the HPLC chromatogram of this separation. As can be observed, the separation has two peaks that are not significantly separated as one peak appears at a retention time 62.38 minutes (Sulfoxide A) and the second peak appears at 63.12 minutes (Sulfoxide B). This method is sufficiently accurate to determine the ratio of isomers, but not separate Sulfoxide A and Sulfoxide B on a preparative scale.

### Example 2

### Chiral HPLC Method

The separation was performed on an Agilent Technologies Mod. 1100 liquid chromatograph, equipped with a chiral column, amylose tris(3,5-dimethylphenylcarbamate) (250 mm×4.6 mm) coated silica gel having a 5 μm particle size (CHIRALPAK AD-H, CHIRAL). Two mobile phases were used: the first mobile phase was n-hexane, and the second mobile phase was 1-propanol. The flow rate of eluant was set to 0.9 ml/minute, and the column temperature was set to 30° C. The test samples contained 50 mg of fulvestrant diluted with 50 ml of a mixture of n-hexane/1-propanol in a ratio of 85:15 by volume. The injection volume was 10 μl.

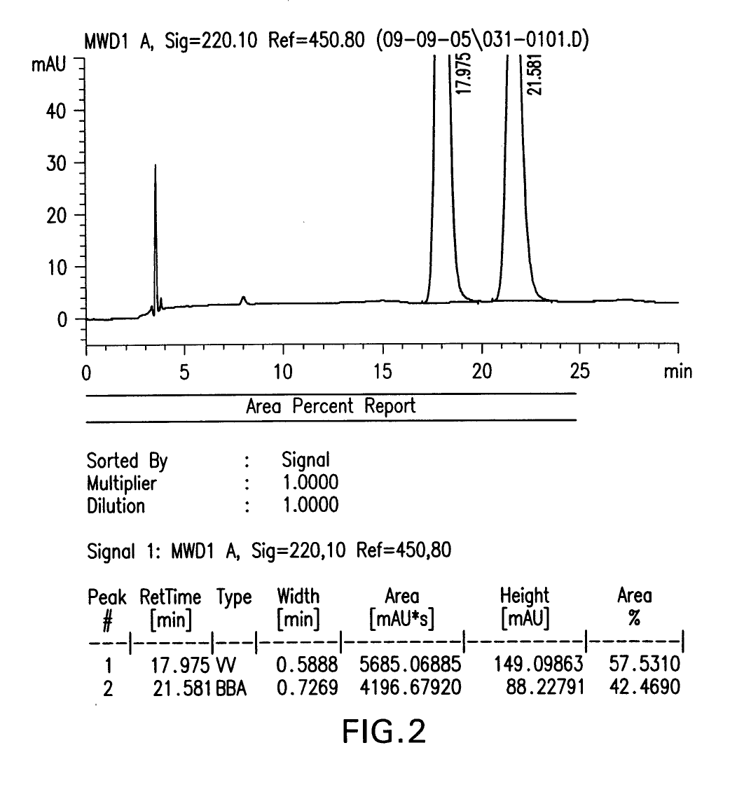

A mixture of 85% of the first mobile phase and 15% of the second mobile phase was pumped through an isocratic system for 45 minutes (i.e., from time 0 to time 45 minutes). The HPLC was equipped with a DAD detector at λ=220 nm. FIG. 2 illustrates the separation using the chiral column. The retention time of the fulvestrant sulfoxide A was 17.97 min; and the retention time of the fulvestrant sulfoxide B was 21.58 min.

### Example 3

### Chiral Preparative HPLC Method

The separation was performed on an Agilent Technologies Mod. 1100 liquid chromatograph, equipped with a chiral column, amylose tris(3,5-dimethylphenylcarbamate) (250 mm×4.6 mm) coated silica gel having a 5 μm particle size (CHIRALPAK AD-H, CHIRAL). Two mobile phases were used: the first mobile phase was n-hexane, and the second mobile phase was 1-propanol. The flow rate of the eluant phase was set to 0.75 ml/minute, and the column temperature was set to 35° C. The test samples contained 5 mg/ml of fulvestrant diluted with a mixture of n-hexane/1-propanol 85:15 (v/v). The injection volume was 600 μl.

A mixture of 85% of the first mobile phase and 15% of the second mobile phase was pumped through an isocratic system for 30 minutes (i.e., from time 0 to time 30 minutes). The HPLC was equipped with a DAD detector at λ=220 nm and 240 nm. The retention time of the fulvestrant sulfoxide A was 17.9 min; and the retention time of the fulvestrant sulfoxide B was 21.2 min. The fractions were collected with automatic device every 0.5 minutes.

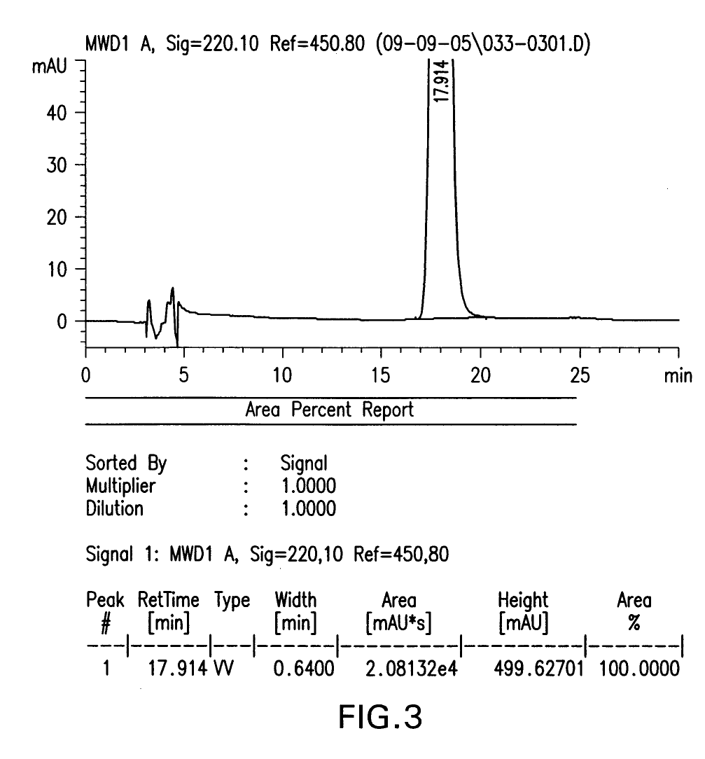

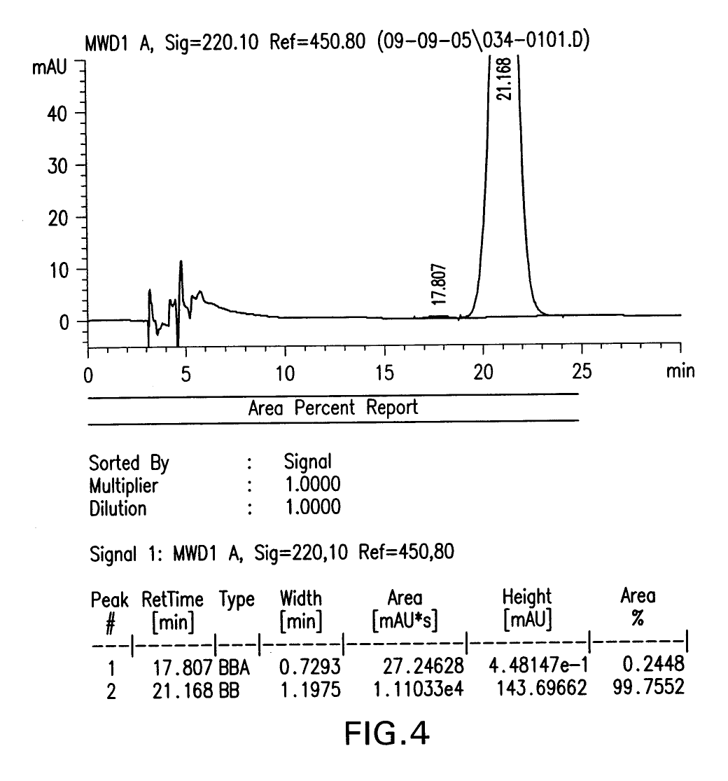

The fractions containing the fulvestrant sulfoxide A were collected and the solvent removed by evaporation using a rotary evaporator to obtain a residual oil. The fractions containing the fulvestrant sulfoxide were collected and the solvent removed by evaporation using a rotary evaporator to obtain a residual oil. The two oils were analyzed by an RP HPLC analytical method applied for the purity control of fulvestrant API, which showed an HPLC purity of >99.9% for both the isomers. In this example, the separation is complete as FIGS. 3 and 4 illustrate HPLC chromatograms for each isomer. FIG. 3 illustrates an HPLC chromatogram for Sulfoxide A and FIG. 4 illustrates a chromatogram for Sulfoxide B. The analytical method is reported in the table below:

| | |
|---|---|
| Instrument | Agilent Technologies Mod. 1100 liquid |
| | chromatograph or equivalent |
| Column & Packing | Zorbax SB-C8, 3.5 μm, 150 × 4.6 mm |
| | (Agilent Technologies, Part. No. 863953-906) |
| | or equivalent |
| Mobile Phase A | H₃PO₄ 0.05% in Water |
| Mobile Phase B | Acetonitrile |

| | | | |
|---|---|---|---|
| Gradient | | Mobile | Mobile |
| | Time | Phase A | Phase B |
| | (min) | (%) | (%) |
| | 0 | 47 | 53 |
| | 5 | 47 | 53 |
| | 30 | 40 | 60 |
| | 60 | 0 | 100 |
| | 80 | 0 | 100 |

| | |
|---|---|
| Run time | 80 minutes |
| Post time | 10 minutes |
| Flow Rate | 1.0 mL/min |
| Detector | λ = 220 nm |
| Column temperature | 40° C. |
| Injection Volume | 10 μL |
| Diluent | Methanol/Acetonitrile 50:50 (v/v) |

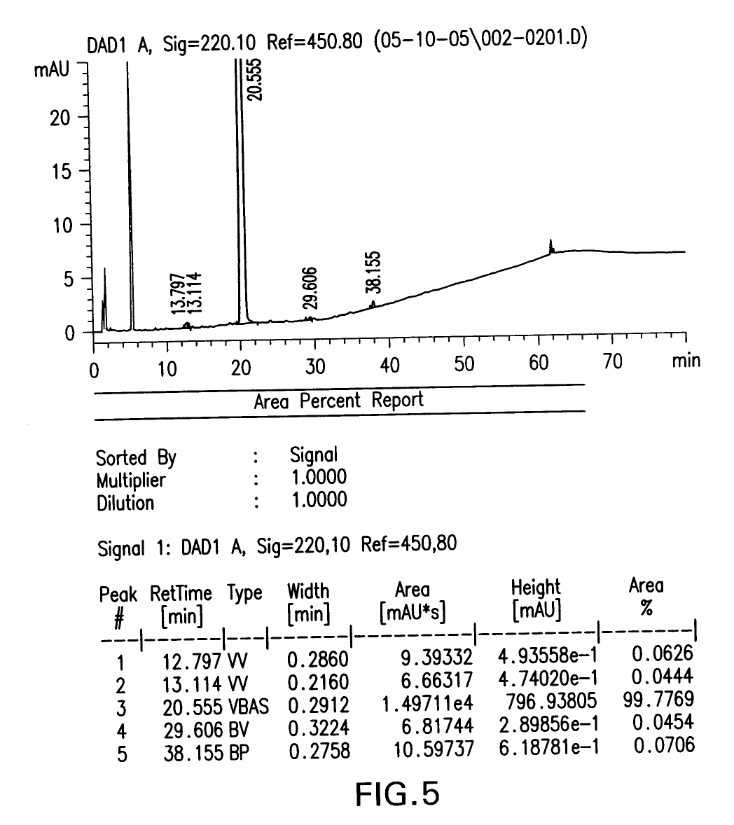

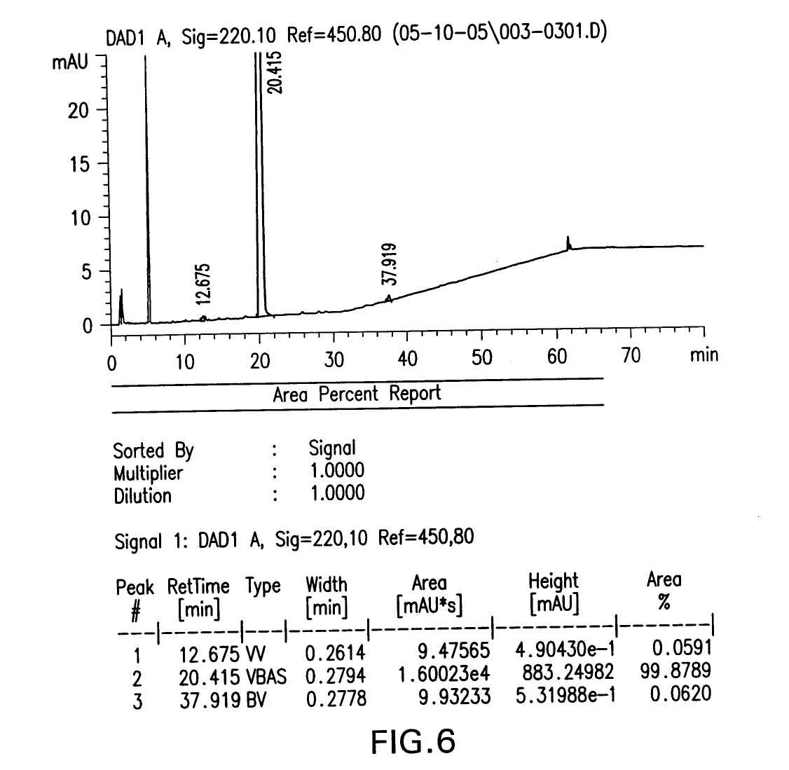

Using the conditions of Example 1, an HPLC chromatogram for each isomer was obtained. If present, the HPLC conditions of Example 1 can illustrate the presence of the second isomer; however, the chromatograms include only one isomer. FIG. 5 illustrates the chromatogram for Sulfoxide A and FIG. 6 illustrates the chromatogram for Sulfoxide B.

### Example 4

### Crystallization of Diastereomerically Pure Fulvestrant Sulfoxide A

The two diastereoisomers residuals were separately crystallized or precipitated with an organic solvent, such as ethyl acetate or toluene, and the two solid diastereoisomers were collected by filtration.

The two oily residuals were submitted alternatively to a treatment with ethyl acetate (4 ml for 0.4 g of residual). The treatment included heating the mixture to reflux temperature until dissolution followed by cooling to 4° C. for 24 hours. The solids were collected by filtration. Alternatively, the solids were treated with toluene (4 ml for 0.4 g of residual) at room temperature, which lead to an immediate precipitation, which was completed after 24 hours at 4° C. The solid Fulvestrant Sulfoxide A and Fulvestrant Sulfoxide B were analyzed by NMR and XDR for the determination of the crystalline structure and the absolute configuration.

### Example 5

### Chiral HPLC Method

The separation of a mixture of fulvestrant isomers was performed on an Waters 600 E liquid chromatograph, equipped with a chiral column, cellulose tris(3,5-dimethylphenylcarbamate) (250 mm×4.6 mm) coated silica gel having a 10 μm particle size (CHIRALPAK OD, DAICEL). Two mobile phases were used: the first mobile phase had n-hexane, and the second mobile phase had 2-propanol. The flow rate of eluant was set to 1.0 ml/minute, and the column temperature was set to 25° C.

The test samples contained 67 mg of fulvestrant diluted with 50 ml of a mixture of n-hexane/2-propanol in a ratio of 85:15 by volume. The injection volume was 5 μl. A mixture of 85% of the first mobile phase and 15% of the second mobile phase was pumped through an isocratic system for 20 minutes (i.e., from time 0 to time 20 minutes). The HPLC was equipped with a PDA detector at λ=210 mm.

After running the sample through the HPLC, each isomer was separated. The retention time of the fulvestrant sulfoxide A was 10.1 min; and the retention time of the fulvestrant sulfoxide B was 11.7 min.

What is claimed is:

1. A method of detecting fulvestrant diastereomers comprising
placing a fulvestrant sample on a HPLC using a reverse phase system;
eluting the sample with two mobile phases using a non-linear gradient having a first mobile phase and a second mobile phase; and
detecting the separate isomers by HPLC,
wherein the first mobile phase is water or an aqueous buffer and the second mobile phase is acetonitrile, tetrahydrofuran, or methanol.

2. The method according to claim 1, wherein the fulvestrant sample is a mixture of fulvestrant sulfoxide A and fulvestrant sulfoxide B.

3. The method according to claim 2, wherein the fulvestrant sample is a racemic mixture or a mixture enhanced in either fulvestrant sulfoxide A and fulvestrant sulfoxide B.

4. The method according to claim 1, wherein the packing material of the reverse phase column is C8 (octyl), C18 (octadecyl), phenyl, pentafluorophenyl, or phenylhexyl.

5. The method according to claim 1, wherein the packing material of the reverse phase column is C8 (octyl) or C18 (octadecyl).

6. The method according to claim 1, wherein the first mobile phase has an initial amount of about 40% to about 70% by volume, and the second mobile phase has an initial amount of about 30% to about 60% by volume.

7. The method according to claim 1, wherein the first mobile phase has a final amount of about 40% to about 0% by volume, and the second mobile phase has a final amount of about 100% to about 50% by volume.

8. A method of separating fulvestrant diastereomers comprising
placing a fulvestrant sample on a HPLC having a chiral column system;
eluting the sample with two mobile phases using an isocratic solvent system having a first mobile phase and a second mobile phase; and
collecting purified fractions of fulvestrant sulfoxide A or fulvestrant sulfoxide B from the column,
wherein the first mobile phase is at least one C₅-C₁₀ alkane and the second mobile phase is a C₃ alcohol.

9. The method according to claim 8, wherein the packing material has the formula:

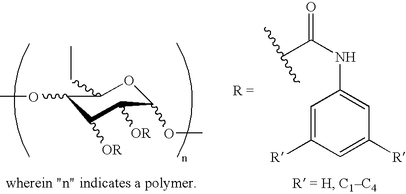

10. The method according to claim 8, wherein the packing material of the chiral column is amylose tris(3,5-dimethylphenylcarbamate), β-cyclodextrin, cellobiohydrolase, selector R-(−)—N-(3,5-dinitrobenzoyl)-phenylglycine, or cellulose tris(3,5-dimethylphenylcarbamate).

11. The method according to claim 8, wherein the packing material of the chiral column is amylose tris(3,5-dimethylphenylcarbamate).

12. The method according to claim 8, wherein the column has a packing particle of a size of about 3 μm to about 10 μm.

13. The method according to claim 8, wherein the column has a packing particle a size of about 5 μm.

14. The method according to claim 8, wherein the first mobile phase is n-hexane, and the second mobile phase is isopropanol.

15. The method according to claim 8, wherein the first mobile phase is present in an amount of about 75% to about 95% by volume and the second mobile phase is present in an amount of about 5% to about 25% by volume.

16. The method according to claim 8, wherein the first mobile phase is present in an amount of about 85% by volume and the second mobile phase is present in an amount of about 15% by volume.

17. The method of claim 8 further comprising crystallizing fulvestrant sulfoxide A or fulvestrant sulfoxide B from the purified fractions by dissolving fulvestrant sulfoxide A or fulvestrant sulfoxide B in organic solvent to form a mixture and precipitating from the mixture fulvestrant sulfoxide A or fulvestrant sulfoxide B.

18. The method according to claim 17, wherein the organic solvents is ethyl acetate or toluene.

19. The method according to claim 17, wherein the mixture is heated to reflux followed by cooling to a temperature of about 0° C. to about 25° C.

20. The method according to claim 19, wherein the mixture is cooled to a temperature is about 4° C.

21. The method according to claim 8, wherein the fulvestrant sulfoxide A or fulvestrant sulfoxide B is 99.5% pure as determined by HPLC.
